(19) United States
(12) Patent Application Publication
(10) Pub. No.: US 2026/0283914 A1
(43) Pub. Date: Sep. 24, 2026

(54) MICROCAPSULES AND ENCAPSULATION THEREOF

(71) Applicant: S H KELKAR AND COMPANY LIMITED, Maharashtra (IN)

(72) Inventors: Abhijit SAHA, Maharashatra (IN); Vijay PATIL, Maharashatra (IN); Avani MAINKAR, Maharashatra (IN); Kedar VAZE, Maharashatra (IN)

(21) Appl. No.: 18/998,085

(22) PCT Filed: Jun. 1, 2023
(86) PCT No.: PCT/IB2023/055640

§ 371 Date: Jan. 23, 2025

(30) Foreign Application Priority Data
Jul. 25, 2022 (IN) 202221042540

(51) Int. Cl. A61K 8/11; A61K 9/50; A61Q 5/06; A61Q 19/00

(52) U.S. Cl. CPC A61K 8/11; A61K 9/5026; A61K 9/5089; A61Q 5/06; A61Q 19/00

(57) ABSTRACT

The invention relates to a polymeric shell and lipophilic active-core material based microcapsule with improved thermal stability.

### FIELD OF INVENTION

The invention relates to a formaldehyde-free polymeric shell and lipophilic active-core material based microcapsules, with improved thermal stability. The microcapsules with cross-linked acrylate vinyl copolymer shell imparts benefits of high heat stability that allows the microcapsule shell to break only at a temperature which is above 250° C., and the shell prevents leaching of core for a longer time at higher temperature (120° C. for 30 minutes). This enables the microcapsules to be suitable for applications at high temperatures along with applications at ambient conditions. The present invention also relates to the process of manufacturing microcapsules.

### BACKGROUND ART

Most live on and rinse off formulations including cosmetic formulations contain fragrances or perfumes in order to confer a pleasant smell to the formulation itself or to the surface, be it textile, skin or hair, onto which the formulation is applied. The fragrances or perfumes are often compounds which are sensitive to various chemicals and to oxidation and hence the need for encapsulation. Also consequently, unwanted interactions with other ingredients of the formulations such as surfactants may lead to an alteration of the fragrance note. In addition, fragrances or perfumes are mostly highly volatile. As a result, a large part of the quantity of fragrance originally added to the formulations gets volatile before the time of application and the remaining quantity of fragrance actually applied onto the treated surface also evaporates within a short time. To overcome these problems, it has already been proposed to incorporate the fragrances or perfumes in microcapsules into the formulations. These microcapsules enable the valuable fragrance or perfume to be distributed relatively homogeneously in a formulation, without having to expose it to the other constituents during storage. Suitable selection of the shell of the capsule also allows effects to be achieved in this way such as retarded release or release on demand upon rubbing or release at higher temperature.

WO2014/189980 A1 describes a population of encapsulated benefit agents having a population diameter coefficient of variation from 6% to 50, preferably from 8% to 35, more preferably from 12% to about 25, said population of encapsulated benefit agents comprising encapsulated benefit agents having a mean diameter of from 3 micrometers to 300 micrometers, preferably from 5 micrometers to 240 micrometers, more preferably from 10 micrometers to 120 micrometers, said encapsulated benefit agent comprising a core and a shell that encapsulates said core, said shell comprising a polymer, preferably a film forming polymer, said shell having a thickness of from 0.5 micrometers to 15 micrometers, preferably from 1 micrometer to 8 micrometers, more preferably from 1.5 micrometers to 6 micrometers and a shell thickness coefficient of variation from 2% to 30, preferably from 4% to 25, more preferably from 6% to 20. Also encompasses a population of encapsulated benefit agents according to claim 1 wherein said shell material comprises, poly(vinyl alcohol), poly(vinyl acetate), poly(vinyl pyrrolidone), poly(vinyl acetate phthalate), vinyl acetate neodecanoic acid co-polymer, vinyl acetate ethylene co-polymer, vinyl acetate crotonic acid neodecanoate co-polymer, vinyl acetate crotonic acid co-polymer, vinyl acetate butyl maleate co-polymer, cellulose acetate, cellulose acetate phathalate, ethyl cellulose, hydroxyl propyl methyl cellulose phathalate, cellulose acetate butyrate, vinyl pyrrolidone vinyl acetate co-polymer, poly(styrene-comaleic acid) isobutyl ester, poly(styrene-co-butadiene), poly(styrene-co-acrylic) and mixtures thereof, but does not relate to microcapsules with high thermal stability.

WO2012/162742 (also published as AU2011902127, EP2714817, CN104053729, U.S. Pat. No. 9,339,781B2, AU2012262664B2, NZ618219, or CA2837897) teaches a method of preparing an aqueous dispersion of polymer encapsulated particulate material, the method comprising: providing a dispersion of the particulate material in a continuous aqueous phase, the dispersion comprising ethylenically unsaturated monomer and a stabiliser for the particulate material; and polymerising the ethylenically unsaturated monomer by non-living free radical polymerisation to form polymer that encapsulates the particulate material, thereby providing the aqueous dispersion of polymer encapsulated particulate material; wherein polymerisation of the ethylenically unsaturated monomer comprises:
(a) polymerising a monomer composition that includes ionisable ethylenically unsaturated monomer so as to form a base responsive water swellable non-living polymer layer that encapsulates the particulate material; and
(b) polymerising a monomer composition that includes non-ionisable ethylenically unsaturated monomer so as to form an extensible, water and base permeable non-living polymer layer that encapsulates the base responsive water swellable polymer layer.

WO2012/162742 also teaches polymer encapsulated particulate material attained, the particulate material being encapsulated by a base responsive water swellable non-living polymer layer comprising polymerised residue of ionisable ethylenically unsaturated monomer, wherein the base responsive water swellable non-living polymer layer is encapsulated by an extensible, water and base permeable non-living polymer layer that comprises polymerised residue of non-ionisable ethylenically unsaturated monomer, but does not relate to microcapsules with high thermal stability.

WO2017004339 (A1) teaches a consumer product comprising a composition, the composition comprising: an adjunct material; a first population of microcapsules, the first population having a first median volume weighted particle size and comprising microcapsules comprising a partitioning modifier and a first perfume oil at a first weight ratio; and a second population of microcapsules, the second population having a second median volume weighted particle size and comprising microcapsules comprising the partitioning modifier and a second perfume oil at a second weight ratio; wherein the first weight ratio and the second weight ratio are different, and/or the first median volume weighted particle size and the second median volume weighted particle size are different; wherein the composition is a fabric and home care composition, thus relates to consumer product with two different microcapsule population and does not teach microcapsules with high thermal stability.

WO2005/002719 teaches a method for preparing microcapsules comprising the steps of: (a) mixing a free-radically polymerizable and ethylenically unsaturated monomer, an emulsifier, an ultrahydrophobe, a hydrophobic material, an initiator and deionized water, to prepare a miniemulsion; and (b) polymerizing the miniemulsion to prepare the microcapsules but does not enable microcapsules with high thermal stability.

US2012076843 (A1) teaches a microcapsule comprising a capsule core and a capsule wall obtainable by a process comprising the free-radical polymerization of an oil-in-water emulsion which comprises the following constituents: 30 to 90% by weight based on the total weight of the monomers of one or more monomers (monomers I) from the group comprising C1 C24-alkyl esters of acrylic acid and/or methacrylic acid acrylic acid methacrylic acid maleic acid fumaric acid and itaconic acid; 10 to 70% by weight based on the total weight of the monomers of one or more ethylenically unsaturated crosslinkers (monomers II) where at least 10% by weight based on the total weight of the monomers I II and III is a highly branched polymeric crosslinker; 0 to 30% by weight based on the total weight of the monomers of one or more monounsaturated monomers (monomer III) which are different from the monomers I and a hydrophobic core material. But it does not teach microcapsules with high thermal stability.

WO2019/121736 teaches an encapsulated perfume composition comprising at least one core-shell microcapsule suspended in a suspending medium, wherein said at least one core-shell microcapsule comprises a core containing at least one perfume ingredient, and a shell surrounding or at least partially surrounding the core, wherein the shell comprises a thermosetting resin formed by the reaction of shell-forming materials selected from monomers, pre-polymers and/or pre-condensates, and wherein the encapsulated perfume composition comprises a polymeric stabilizer that is the reaction product of a polymeric surfactant and a silane containing a functional group capable of forming covalent bonds with the shell. But the thermal stability of the microcapsule composition as per this prior art is not disclosed.

WO2020190689 (also published as US 2020/0315931 A1) teaches a population of microcapsules comprising a capsule core and a capsule shell, the capsule shell being hydrolysable, the microcapsules made by an oil-in-water microencapsulation process comprising: a) dispersing in an aqueous phase a polymeric emulsifier and optionally, an initiator; b) dispersing in one or more oil phases: i) an initiator, and a core material, ii) a first multifunctional (meth)acrylate monomer having greater than one ester group on average in the monomer and having a hydrophilicity index of less than 20, iii) a second multifunctional (meth)acrylate monomer, the second multifunctional (meth)acrylate comprising a hydrophilic multifunctional polar monomer having a hydrophilicity index of at least 20 and said second multifunctional polar monomer comprising 50% or less of the capsule shell, wherein the first and second multifunctional (meth)acrylate monomers together comprise greater than 80% by weight of the capsule shell, iv) an acidic (meth)acrylate monomer or at least one oil soluble or dispersible simple acid or both, the acidic (meth)acrylate monomer having one or more groups which are selected from carboxy and sulfonic groups, and v) optionally from 0 to 50% by weight of an aliphatic polyester, the aliphatic polyester having two or more of acrylate or methacrylate groups; c) emulsifying the one or more oil phases into the water phase under high shear agitation to form an oil-in-water emulsion comprising droplets of the core material and oil phase monomers dispersed in the water phase; d) activating the initiator or initiators by heat or actinic radiation to react the monomers and optional aliphatic polyester thereby forming a capsule shell which is polymeric, surrounding the droplets of the emulsion. Thus, this prior art teaches multifunctional (meth)acrylate monomers as the shell wall variant that is free of involving vinyl acetate monomer but involving polyvinyl pyrrolidone together with butyl acrylate going into the microcapsule shell formation does not relate to capsules with increased thermal stability.

CN109453724A discloses a preparation method of a sustained-release microcapsule with a multi-core interior. The preparation method comprises the following steps: dispersing a suspension mixed with an acrylate polymer, a volatile organic solvent, liquid essence and 25 porous starch into a colloid-protect reagent aqueous solution by mechanical agitation to form an oil-in-water system; then performing pressure reduction to remove the volatile organic solvent in the oil-in-water system, and conducting interfacial phase separation on the acrylate polymer, the liquid essence and the porous starch to form an acrylate polymer microcapsule wrapped with the liquid essence and the porous starch in the interior; and 30 further adding an ethylene glycol dimethacrylate prepolymer, and performing heating curing to obtain a crosslinked acrylate polymer essence microcapsule with a multi-core interior, namely the sustained-release microcapsule with the multi-core interior. The method is simple to operate and efficient for preparation, and wrapped essence can be released gradually, is lasting in fragrance, and can be widely applied in the fields of cosmetics, household supplies or personal care products and functional materials.

US20170211019 (also published as WO 2017/132101A1, JP 6651637B2, or EP3408363A1) teaches a composition comprising, based upon total composition weight: a) from about 0.01% to about 1%, of a polymeric material comprising a first polymer and a second polymer; said first polymer is derived from the polymerization of from about 5 to about 100 mole percent of a cationic vinyl addition monomer, from about 0 to about 95 mole percent of a non-ionic vinyl addition monomer, from about 50 ppm to about 1,950 ppm of a cross-linking agent comprising two or more ethylenic functions, about 0 ppm to about 10,000 ppm chain transfer agent; said second polymer being derived from the polymerization of from about 5 to about 100 mole percent of a cationic vinyl addition monomer, from about 0 to about 95 mole percent of a non-ionic vinyl addition monomer, from about 0 ppm to about 45 ppm of a cross-linking agent comprising two or more ethylenic functions, about 0 ppm to about 10,000 ppm chain transfer agent; b) from about 0% to about 35% of a cationic quaternary fabric softener active material, the iodine value of the parent fatty acyl compound or acid from which the alkyl or, alkenyl chains are derived being from about 5 to about 60; and c) a population of perfume microcapsules with the proviso that said population of perfume microcapsules comprises a microcapsule wall material comprising one or more polyacrylate polymers; said composition being a fabric and home care product. The polymer is derived by involving-(iii) an anionic monomer selected from the group consisting of acrylic acid, methacrylic acid, itaconic acid, crotonic acid, maleic acid, fumaric acid, as well as monomers performing a Sulfonic acid or phosphonic acid functions, such as 2-acrylamido 2-methyl propane Sulfonic acid, and their salts. The perfume microcapsules comprising a microcapsule wall material essentially involving cationic vinyl addition monomer of quaternary ammonium type also does not teach any improved thermal stability of the microcapsules.

WO 2017123965 also published as US 2019/0054440 A1, AU2017207981 B2, EP3402674, CN108778730B, BR112018014242, US20190054440, JP2019505375 (JP 6938514B2), CA3011107, IN201817026022, MX2018008726 teaches A microcapsule comprising: i. a lipophilic core material, and ii. a microcapsule shell; wherein said microcapsule shell is formed from oil-in-water emulsion polymerisation of a monomer mixture consisting essentially of: (a) greater than 70 to about 99% by weight of at least one polyfunctional ethylenically unsaturated monomer, (b) about 1 to about 30% by weight of at least one unsaturated carboxylic acid monomer or its ester, and (c) about 0 to about 30% by weight of at least one vinyl monomer.

WO2017040759 also published as CA2980193, CN107530672, AU2016317844, EP3344382, teaches an aqueous slurry composition, comprising an aqueous medium having dispersed therein oily medium-containing microcapsules, wherein the oily medium-containing microcapsules comprise an ionic acrylate copolymer shell encapsulating said oily medium. Thus teaches only acrylic polymer based microcapsules and does not involve any shell structure based on meth (acrylic)-vinyl acetate copolymer to attain thermally stable microcapsules through a distinct process.

EP2397120B2 also published as WO 2011/158962, ES2597980, U.S. Pat. No. 9,464,263B2, CN102946843, BR112012032063, JP2013530253, MX344969 teaches a liquid consumer product having a density in the range from 0.900 g/cm3 to 1.400 g/cm³, preferably from 0.900 g/cm3 to 1.250 g/cm³, and which comprises core shell microcapsules wherein:
the microcapsule shell is formaldehyde-free and is made of starting materials such that 50%-100% by weight of said materials have a density equal to or less than 1.05 g/cm³;
the microcapsule core contains a fragrance composition, which composition comprises:
a) 20-100% by weight of at least one cyclic fragrance material with a density greater than 0.950 g/cm³ and a ClogP in the range from 1.00 to 6.00;
b) 0-50% by weight of at least one oil soluble organic compound having a density greater than 0.950 g/cm³;
c) 0-80% by weight of at least one material selected from cyclic fragrance ingredients with densities equal to or less than 0.950 g/cm³ and non-cyclic fragrance ingredients with densities which may be greater or less than 0.950 g/cm³;
where the sum of a), b) and c) equals 100%, wherein the weight ratio of core materials to shell materials is in the range from 50:1 to 1:1; and wherein the said starting materials comprise at least 50% by weight, preferably at least 60% by weight, of (meth)acrylic acid and/or (meth)acrylates; wherein the dosage of the microcapsules into the liquid consumer product is in the range from 0.01 to 10% by weight, preferably from 0.05% to 2.5% by weight, more preferably from 0.1 to 1.25% by weight of the liquid product composition, and hence teaches select density based shell wall material to encapsulate select fragrance which shell material does not involve any poly((meth)acrylate-co-vinyl acetate copolymer to lead to thermally stable crosslinked microcapsules based on a select process of polymerization.

WO2014032920A1, EP2890486B1, CN104755162B, KR1020150052046, RU2015111081, BR112015004387, ID2016/05358, RU0002639909, JP2015535858, CA2882427, 1199/CHENP/2015; MX2015002649, JP2017105791; teaches a microcapsule comprising a core of hydrophobic material composed of at least one fragrance and a microcapsule shell obtainable by the suspension polymerization of the following monomers:
(a) one or more C1-C₂₄-alkyl ester(s) of (meth)acrylic acid (monomer A),
(b) one or more bi- or polyfunctional monomers (monomer B) and
(c) optionally, one or more other ethylenically unsaturated monomers (monomer C),
wherein the shear rate for the preparation of the emulsion lies in the range of from 150 to 500 rpm, the stirring time for the preparation of the emulsion lies in the range of from 15 min to 180 min and an anchor-type stirring blade or a MIG-stirrer is used for the preparation of the emulsion, however does not teach any thermally stable microcapsules that can be attainable out of specific involvement of vinyl acetate monomer by avoiding the shear rates mentioned in this prior art.

WO2017/004339, US20170002301, CA2989002, CN107835681, EP3316854 B1, JP2018522976, PL3316854, IN201717045571, MX364218, JP2020073672; teaches a consumer product comprising a composition, the composition comprising: an adjunct material; a first population of microcapsules, the first population having a first median volume weighted particle size and comprising microcapsules comprising a partitioning modifier and a first perfume oil at a first weight ratio; and a second population of microcapsules, the second population having a second median volume weighted particle size and comprising microcapsules comprising the partitioning modifier and a second perfume oil at a second weight ratio; wherein the first weight ratio and the second weight ratio are different, and/or the first median volume weighted particle size and the second median volume weighted particle size are different; wherein the composition is a fabric and home care composition, and hence this prior art does not teach that by avoiding partitioning modifiers and based on only one type of microcapsules with only one fragrance type thermally stable microcapsules could be attained.

While microcapsules encapsulating actives are known in the art, there is still a need to explore for microcapsule that would have lipophilic material as core such as fragrance that would be thermally stable to break down at above 250° C. to suit high temperature applications including steaming of fabric, hair-straightening, paints, textile-processing, shoe insole making.

### OBJECTS OF THE INVENTION

It is thus one object of the present invention to provide for microcapsules with lipophilic core that would be thermally stable as microcapsules so that during their applications at higher temperature the shell doesn't break and thus prevent the loss of the lipophilic core and under any mechanical stress the shell would break to release the core/benefiting agent.

It is another object of the present invention to provide for said microcapsule with lipophilic core including liquid active such as fragrance.

It is yet another object of the present invention to provide for said microcapsules synthetic process that would involve preheating/pre-polymerization of monomers/shell materials in oil phase to be yet carried out under a preparation procedure involving a single polymerization step; i.e. all the reactive monomers are copolymerize together in one step.

### DESCRIPTION OF THE INVENTION

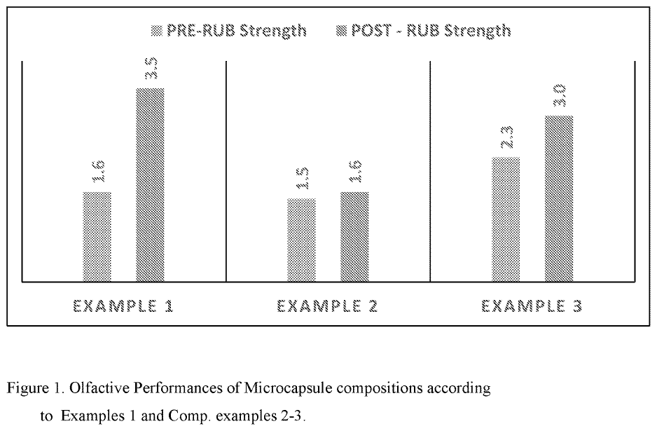

FIG. 1 shows the Olfactive Performances of Microcapsule compositions.

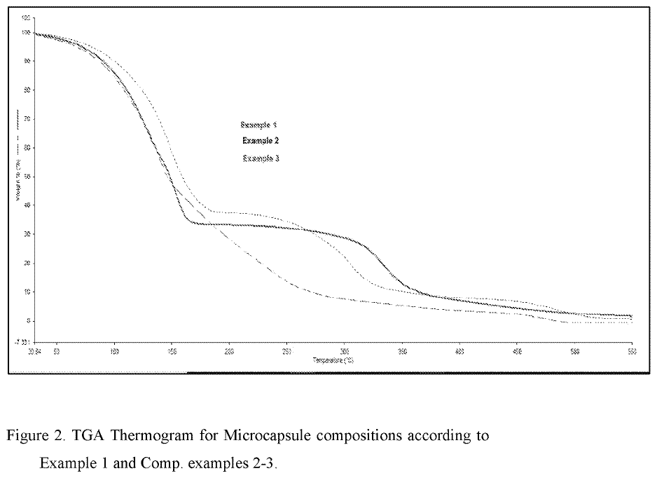

FIG. 2 shows the TGA Thermogram for Microcapsule compositions.

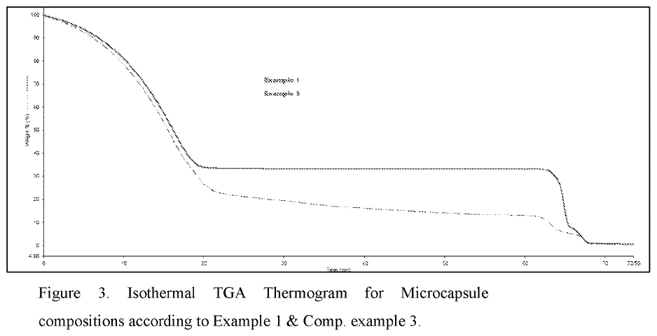

FIG. 3 shows the Isothermal TGA Thermogram for Microcapsule compositions.

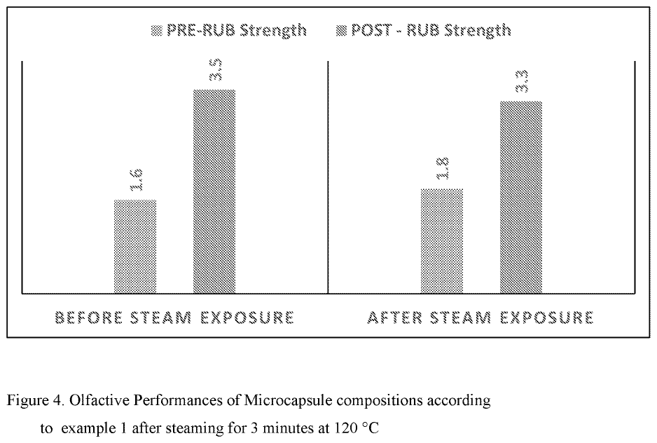

FIG. 4 shows the Olfactive Performances of Microcapsule compositions after steaming for 3 minutes at 120° C.

The present invention relates to a microcapsule comprising a lipophilic core material and a microcapsule shell wherein said microcapsule shell is formed from oil-in-water emulsion polymerisation of a mixture of monomers, more than 50 percent by weight of the mixture of monomers consisting in monomers having a density higher than 1.05, said mixture of monomers comprising a) more than 30 percent by weight of one or more of ethylenically unsaturated acid monomer(s) based on the total weight of the mixture of monomers, b) one or more monofunctional acrylate and/or methacrylate monomer(s), c) one or more multifunctional acrylate and/or methacrylate monomers, and d) vinyl acetate monomer.

In an embodiment the microcapsule is thermally stable at 250° C.

In an embodiment the microcapsule is formaldehyde-free.

In an embodiment the microcapsule is characterised in that the log P values of all the monomers comprised in the mixture of monomers range from 0.5 to 4.0.

In an embodiment the microcapsule is characterised in that the ethylenically unsaturated acid monomer(s) is selected from acrylic acid, methacrylic acid, crotonic acid, 2-carboxyethyl acrylate, glutaconic acid, 3,3-Dimethylacrylic acid, itaconic acid, maleic acid, fumaric acid or a mixture of two or more of said acids.

In an embodiment the microcapsule is characterised in that the ethylenically unsaturated acid monomer is methacrylic acid.

In an embodiment the microcapsule is characterised in that the concentration of ethylenically unsaturated acid monomers in the monomer mixture is equal or inferior to 45 percent by weight based on the total weight of the mixture of monomers.

In an embodiment the microcapsule is characterised in that the concentration of ethylenically unsaturated acid monomers in the monomer mixture is comprised between 30 and 45 percent by weight based on the total weight of the mixture of monomers.

In an embodiment the microcapsule is characterised in that the monofunctional acrylate and/or methacrylate monomer(s) is selected from polymerizable molecules with one ester functionality of the following formula

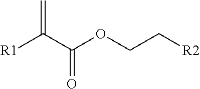

wherein
R1═H/CH₃,
R2═—OH, —(CH₂)ₙ—OH, O—CH₃, —O—(CH₂)ₘ—OH, —O—(CH₂)ₙ—CH₃, —(O—CH₂—CH₂)ₙ—OH, —(O—CH₂—CH₂—CH₂)ₙ—OH, —(O—CH₂—CH₂)ₙ—O—CH₃, —(O—CH₂—CH₂)ₙ—O—CH₂—CH₃, —(O—CH₂—CH₂—CH₂)ₙ—O—CH₂—CH₃, —(O—CH₂—CH₂—CH₂)ₙ—O—CH₃,
(O—CH₂—CHR3)ₙ—CH₃
n=1 to 10, m=2 to 10, and R3=methyl or ethyl,
or a mixture of two or more of said monomers.

In an embodiment the microcapsule is characterised in that the monofunctional acrylate and/or methacrylate monomer(s) is selected from 2-hydroxyethyl methacrylate, poly(ethylene glycol) methacrylate, Poly(propylene glycol) methacrylate, 4-hydroxybutyl acrylate, hydroxybutyl methacrylate, Hydroxypropyl acrylate, Hydroxypropyl methacrylate 6-hydroxyhexyl acrylate, 6-hydroxyhexyl methacrylate, or a mixture of two or more of said monomers.

In an embodiment the microcapsule is characterised in that the monofunctional acrylate and/or methacrylate monomer(s) is hydroxyethyl methacrylate.

In an embodiment the microcapsule is characterised in that the concentration of the monofunctional acrylate and/or methacrylate monomer(s) is comprised between 5 and 50 percent by weight based on the total weight of the mixture of monomers.

In an embodiment the microcapsule is characterised in that the multifunctional acrylate and/or methacrylate monomer(s) is selected from polymerizable molecules with more than one ester functionalities.

In an embodiment the microcapsule is characterised in that the one or more multifunctional acrylate and/or methacrylate monomer(s) is a mixture of two or more of said monomers,

In an embodiment the microcapsule is characterised in that the multifunctional acrylate and/or methacrylate monomer(s) is selected from ethylene glycol dimethacrylate, neopentyl glycol dimethacrylate, 1,4-Butanediol diacrylate, 1,4-Butanediol dimethacrylate, 1,6-hexane diol dimethacrylate, Glycerol diacrylate, Glycerol dimethacrylate, 1,10-Decanediol dimethacrylate, Bis [2-(methacryloyloxy)ethyl]phosphate, pentaerythritol triacrylate, trimethylolpropane triacrylate, trimethylolpropane trimethacrylate, pentaerythritol tetraacrylate, or a mixture of two or more of said monomers.

In an embodiment the microcapsule is characterised in that the multifunctional acrylate and/or methacrylate monomer(s) is a mixture of two or more of said monomers, each of said monomer representing less than 30 percent by weight based on the total weight of the mixture of monomers.

In an embodiment the microcapsule is characterised in that the total concentration of the multifunctional acrylate and/or methacrylate monomer(s) in the monomer mixture is less than 60 percent by weight based on the total weight of the mixture of monomers.

In an embodiment the microcapsule is characterised in that the concentration of the multifunctional acrylate and/or methacrylate monomer(s) in the monomer mixture is comprised between 20 and 50 percent by weight based on the total weight of the mixture of monomers.

In an embodiment the microcapsule is characterised in that the concentration of vinyl acetate monomer in the monomer mixture is comprised between 0.05 and 15 percent by weight based on the total weight of the mixture of monomers.

In an embodiment the microcapsule is characterised in that the concentration of a) ethylenically unsaturated acid monomer(s), b) monofunctional acrylate and/or methacrylate monomer(s), c) multifunctional acrylate and/or methacrylate monomer(s), and d) vinyl acetate monomer, is at least 95 percent by weight based on the total weight of the mixture of monomers.

In an embodiment the microcapsule is characterised in that the concentration of a) ethylenically unsaturated acid monomer(s), b) monofunctional acrylate and/or methacrylate monomer(s), c) multifunctional acrylate and/or methacrylate monomer(s), and d) vinyl acetate monomer, [a)+b)+c)+d)] is 100 percent by weight based on the total weight of the mixture of monomers.

In an embodiment the microcapsule is characterised in that the Particle Size of the microcapsule ranges from 8 to 35 microns of Dv (90) value.

In an embodiment the microcapsule is characterised in that the lipophilic core material of the microcapsules has a density equal or less than 0.95 g/cm³ at 25° C. with a combined log P comprised between 2.5 and 6.0.

In an embodiment the microcapsule is characterised in that the lipophilic core material of the microcapsules comprises at least 95 percent by weight, preferably 100 percent by weight, based on the total weight of said lipophilic core material, of one or more of the following ingredients: fragrances, profragrances, emollient oils, essential oils, hair-benefitting agents, skin-benefitting agents, conditioner actives, cosmetic care actives, personal care actives, UV absorbers, vitamins, anti-oxidants, anti-microbial agents, anti-viral, flavors, anti-malodor agents, pharmaceutical agents, dyes, printing inks, pesticides, biocides, agrochemicals, coating materials, anti-ageing actives.

In an embodiment the microcapsule is characterised in that the lipophilic core material of the microcapsules comprises one or more of the following ingredients: fragrances, essential oils, hair-benefitting agents, skin-benefitting agents, anti-microbial agents, anti-viral agents, anti-malodor agents.

In an embodiment the microcapsule is characterised in that the lipophilic core material weight divided by the shell weight of the microcapsule is comprised between 15 and 0.2, for example between 15 and 0.33, for example between 15 and 0.4.

Aqueous microcapsule composition comprising water and microcapsules according to claim 1 wherein the water represents from 35 to 82 weight percent of the total weight of the aqueous microcapsule composition.

The present invention also relates to an aqueous microcapsule composition comprising a microcapsule comprising a lipophilic core material and a microcapsule shell wherein said microcapsule shell is formed from oil-in-water emulsion polymerisation of a mixture of monomers, more than 50 percent by weight of the mixture of monomers consisting in monomers having a density higher than 1.05, said mixture of monomers comprising a) more than 30 percent by weight of one or more of ethylenically unsaturated acid monomer(s) based on the total weight of the mixture of monomers, b) one or more monofunctional acrylate and/or methacrylate monomer(s), c) one or more multifunctional acrylate and/or methacrylate monomers, and d) vinyl acetate monomer, and wherein the lipophilic core material represents between 15 and 45 percent by weight of the total weight of the aqueous microcapsule composition.

In an embodiment, the aqueous microcapsule composition is characterized in that it comprises one or more emulsifiers wherein the emulsifiers represent from 0.05 to 5 weight percent of the total weight of the aqueous microcapsule composition.

In an embodiment, the aqueous microcapsule composition is characterized in that the weight of water, microcapsules and emulsifier represents at least 90% by weight of the total weight of the aqueous microcapsule composition.

The present invention also relates to a process for preparing an aqueous microcapsule composition comprising a microcapsule comprising a lipophilic core material and a microcapsule shell
wherein said microcapsule shell is formed from oil-in-water emulsion polymerisation of a mixture of monomers, more than 50 percent by weight of the mixture of monomers consisting in monomers having a density higher than 1.05, said mixture of monomers comprising a) more than 30 percent by weight of one or more of ethylenically unsaturated acid monomer(s) based on the total weight of the mixture of monomers, b) one or more monofunctional acrylate and/or methacrylate monomer(s), c) one or more multifunctional acrylate and/or methacrylate monomers, and d) vinyl acetate monomer, and wherein the lipophilic core material represents between 15 and 45 percent by weight of the total weight of the aqueous microcapsule composition,
comprising the steps of:
   1. dissolving the mixture of monomers together with an initiator in an oil phase comprising the lipophilic core material and heating the oil phase to form prepolymer(s),
   2. dissolving an emulsifier in an aqueous phase,
   3. emulsifying the oil phase of step 1 into the aqueous phase of step 2, and
   4. heating the emulsion from step 3 to form a suspension of core-shell microcapsules in water.

In an embodiment the process for preparing an aqueous microcapsule composition is characterized in that the emulsification step of the core phase in the water phase is obtained by stirring at 500-1500 rpm for up to 12 minutes using a propeller type stirrer.

The present invention also relates to a non-therapeutic method of use of a microcapsule or an aqueous microcapsule composition as claimed comprising employing said microcapsule to deliver the lipophilic core material for industrial compositions that are related to home care products, personal care products, textile products, printing and coating applications products, pharmaceutical formulations products, consumer goods products, and in agro-industrial formulation products.

In an embodiment, said non-therapeutic method of use is characterized in that the mechanical stress and temperature conditions at which the microcapsule is exposed are sufficient to break the microcapsule shell and to deliver the lipophilic core material.

In an embodiment, said non-therapeutic method of use according to the present invention applies to the steaming of fabric, hair-straightening, paints, textile-processing, and shoe insole making.

### Description of Embodiments of the Invention

The following description relates to particular embodiments pertaining to the present invention.

As discussed hereinbefore, the present invention provides for polymeric shell and lipophilic active-core containing formaldehyde-free microcapsules, with high thermal stability, which breaks above 250° C., is suitable for applications at high temperatures such as but not limited to in steaming of fabric, hair-straightening, paints, textile-processing, and shoe insole making etc., where the polymeric shell comprising a crosslinked (meth)acrylic-vinyl acetate copolymer.

For the man skilled in the art, a microcapsule comprising a lipophilic core material and a microcapsule shell which is thermally stable at 250° C. means that said microcapsule shell does not break at 250° C.

Any appropriate measurement method can advantageously be used for measuring said characteristic of being thermally stable at 250° C.

For example, it may advantageously be measured by taking a sample of the microcapsule slurry intended to be commercialized, e.g. a sample of the microcapsule slurry as obtained at the end of the example 1 hereinbelow; by examining said microcapsule slurry sample using a thermogravimetric analyzer (TGA); by subjecting it to a ramp of increased temperatures, for example from ambient temperature up to 250° C. by increments of 30° C. per minute; and by measuring the lipophilic core material weight loss. When lipophilic core material weight loss is below 7.5 percent by weight, preferably below 5 percent by weight based on the total weight of the encapsulated core material, it may be concluded that the microcapsule is thermally stable at 250° C.

For example, if the ambient temperature is 20° C., the duration of the temperature increases up to 250° C. at which the sample is subjected and the corresponding TGA observation will take about 7 minutes and 40 seconds.

Thus, the shell of the encapsulated lipophilic core material microcapsule will only break at a temperature which is above 250° C. when the microcapsules are examined using a thermogravimetric analyzer (TGA).

Advantageously, an additional characteristic of the polymeric shell of the encapsulated lipophilic core material microcapsule of the present invention is that the shell can prevent the loss of lipophilic core materials by leakage while the microcapsules were exposed to 120° C. for a long time, e.g. more than 30 minutes. This further demonstrates the thermal stability of the microcapsules that enables the microcapsules to retain their integrity for a longer time in applications where prolonged thermal exposure is required.

The shell in the present invention comprising off polymers made of building blocks, which are essentially polymerizable molecules, more than 50% of which are having densities of 1.05 g/cm³ or higher at 25° C., and having log P values ranging between 0.5 to 4.0, with ester functionality and ester-forming functionalities.

By definition, Log P refers to the octanol/water partitioning coefficient (P) of any individual ingredient, which is the ratio between its equilibrium concentrations in octanol and in water. The partitioning coefficients of the ingredients are given in the form of their logarithm to the base 10, Log P. Here the individual Log P values are usually provided by the raw materials suppliers. The combined Log P value for the fragrance is determined by averaging the individual Log P values on weight % basis.

By definition, here density refers to individual density of all the materials used and the values are expressed in g/cm³ at 25° C. The values are collected from Sigma Aldrich, India (https://www.sigmaaldrich.com/IN/en) and/or from The Good Scents Company Information System (http://www.thegoodscentscompany.com/). The density of the fragrance in the Example 1-3 was determined using Pycnometer using ATSM D 369 method at 25° C.

The polymerizable molecules in this invention are referred to as the organic molecules are having one or more of ethylenically unsaturated moiety (ies).

By definition, aqueous microcapsule composition or slurry in this invention is referred to as a aqueous medium contains dispersed microcapsules in presence of an emulsifier where the microcapsule contains lipophilic core (e.g., fragrance) and a polymeric shell which is formed by polymerization of ethylenically unsaturated molecules initiated by an oil-soluble (thermal) initiator at high temperature. The end-product of Example 1 is the referred to as the aqueous microcapsule composition or slurry as per this invention. For example, as indicated in the foregoing example 1, the emulsifier is present in the final slurry; the majority of the initiator generates lauric free radicals which then start forming the crosslinked shell polymer and remaining lauric free radicals form lauric acid and thus remain dissolved/entrapped in the core.

By definition, polymerizable molecules with ester functionality is referred to as the ethylenically unsaturated molecules having at least one ester moiety.

By definition, polymerizable molecules with ester-forming functionality is further clarified to as the ethylenically unsaturated molecules having at least one free acid functionality that can be chemically transformed into ester moiety. Examples of such polymerizable molecules with one free acid functionality are such as but not limited to acrylic acid, methacrylic acid, crotonic acid, and 2-carboxyethyl acrylate, Glutaconic acid, 3,3-Dimethylacrylic acid, itaconic acid, maleic acid, fumaric acid etc.

The building blocks in this invention are comprising off (a) more than 30 weight % of total weight of shell of one or more of polymerizable molecules with one free acid functionality.

The building blocks in this application are comprising off (b) one or more of polymerizable molecules with one ester functionality of the following formula

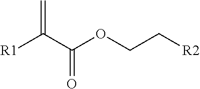

Wherein R¹═H/CH₃,
R2═—OH, —(CH₂)ₙ—OH, O—CH₃, —O—(CH₂)ₘ—OH, —O—(CH₂)ₙ—CH₃, —(O—CH₂—CH₂)ₙ—OH, —(O—CH₂—CH₂—CH₂)ₙ—OH, —(O—CH₂—CH₂)ₙ—O—CH₃, —(O—CH₂—CH₂)ₙ—O—CH₂—CH₃, —(O—CH₂—CH₂—CH₂)ₙ—O—CH₂—CH₃, —(O—CH₂—CH₂—CH₂)ₙ—O—CH₃,
(O—CH₂—CHR3)ₙ—CH₃
n=1 to 10, m=2 to 10,
R3=methyl or ethyl.

Examples of polymerizable molecules with one ester functionality of the above-mentioned formula are such as but not limited to 2-hydroxyethyl methacrylate, poly(ethylene glycol) methacrylate, Poly(propylene glycol) methacrylate, 4-hydroxybutyl acrylate, hydroxybutyl methacrylate, Hydroxypropyl acrylate, Hydroxypropyl methacrylate, 6-hydroxyhexyl acrylate, 6-hydroxyhexyl methacrylate, etc.

The building blocks in this invention are comprising off (c) less than 60 weight % of the shell of two or more of polymerizable molecules with more than one ester functionalities where each of molecules is less than 30 weight % of the shell in the composition of the building blocks.

Examples of polymerizable molecules with more than one ester functionalities such as but not limited to are ethylene glycol dimethacrylate, neopentyl glycol dimethacrylate, 1,4-Butanediol diacrylate, 1,4-Butanediol dimethacrylate, 1,6-hexane diol dimethacrylate, Glycerol diacrylate, Glycerol dimethacrylate, 1,10-Decanediol dimethacrylate, Bis [2-(methacryloyloxy)ethyl]phosphate, pentaerythritol triacrylate, trimethylolpropane triacrylate, trimethylolpropane trimethacrylate, pentaerythritol tetraacrylate, etc.

The building blocks in this application are comprising off (d) vinyl acetate.

The combined weight % of (a), (b), (c), and (d) is 100% to form the shell according to this invention.

The microcapsule of the present invention is obtained by a three-step process involving i) preheating/pre-polymerizing of the building blocks i.e., polymerizable molecules in the lipophilic active core phase in presence of an oil-soluble initiator, ii) emulsifying the core in a water phase, iii) heating the emulsion at high temperatures to form the cross-linked shell polymer.

The detailed process for preparation of microcapsules as per the present invention may advantageously involve the following steps:
1. a) Dissolving the polymerizable molecules (i.e., building blocks) and initiator in lipophilic core i.e., oil phase b) heating the oil phase at 40-55° C. under nitrogen while stirring at 100-200 rpm for 15-45 minutes to form prepolymer(s);
2. Dissolving the emulsifier(s) in water phase;
3. Emulsifying the oil phase of step 1 into the aqueous phase of step 2 by stirring at 500-1500 rpm for up to 12 minutes using a propeller type stirrer;
4. Heating the emulsion under nitrogen at first at 40-60° C. for 15 to 45 minutes and then at 65-85° C. for 4-6 hours at 100-400 rpm;
5. Quenching the unreacted monomers at 65-85° C. by adding an aqueous persulfate solution.

The core of the microcapsules in the invention is comprising off lipophilic active with density equal or less than 0.95 g/cm³ at 25° C. with a combined log P of 2.5-6.0. The core is attributing 15-45 weight % of the aqueous microcapsules composition. Whereas core: shell ratio varies from 15:1 to 1:5; for example from 15:1 to 1:3 or from 15:1 to 2:5. Total solids of the aqueous microcapsule composition varies between 18-65 weight % which includes lipophilic core-polymeric shell microcapsules, emulsifier(s), and other ingredients.

The lipophilic core is comprising of essentially but not limited to fragrances, profragrances, emollient oils, essential oils, hair and skin-benefitting agents, conditioner actives, cosmetic and personal care actives, UV absorbers, vitamins and anti-oxidants, anti-microbial and anti-viral agents, flavors, anti-malodor agents, pharmaceutical agents, dyes and printing inks, pesticides and biocides, agrochemicals, coating materials, anti-ageing actives, etc.

Emulsifier used in this invention are essentially anionic, non-ionic, and cationic small molecules, oligomers, and polymers. Examples of anionic emulsifiers are salts of alkyl sulfates, alkyl ether sulfates, alkyl carboxylates, alkyl succinamates, alkyl sulfosuccinates, alkyl sulfate salts such as sodium dodecyl sulfate, alkyl sarcosinates, alkyl or alkyl ether or alkylaryl ether phosphate esters, ammonium, sodium or potassium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate. Non-ionic emulsifiers used in this invention are but not limited to acetylated monoglycerides, lactylated monoglycerides, phosphated or sulfated tristyrylphenol ethoxylates, secondary alcohol ethoxylates, oligoethyleneglycol esters of fatty acids, lactylated propyleneglycol monoglycerides, sorbitan esters, sorbitan-polyoxyethylene monoglycerides, polyglycerol esters, diacetyltartarate esters of monoglycerides, succinylated esters of monoglycerides. Polymeric emulsifiers used in this application are non-ionic surfactants such as diblock copolymers of polyethylene oxide and polyethylene or polypropylene oxide, poly(styrene sulfonate) sodium salt, isobutylene-maleic anhydride copolymer, gum arabic, sodium alginate, carboxymethylcellulose, cellulose sulfate and pectin, poly(styrene sulfonate), gum arabic, carrageenan, sodium alginate, pectic acid, tragacanth gum, and agar; carboxymethyl starch, phosphated starch, lignin sulfonic acid; polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, and mixtures thereof.

The quantity of the emulsifier ranging between 0.05 to 5 weight % of the microcapsule composition described herein.

In one non-limiting embodiment, the initiator used and dissolved in lipophilic core for preparing the microcapsule is a thermal initiator selected from the group consisting of dibenzoyl peroxide, dioctanoyl peroxide, dilauroyl peroxide, didecanoyl peroxide, tert-butyl peracetate, tert-butyl perlaurate, tert-butyl perbenzoate, dicetyl peroxydicarbonate, peroxydicarbonate, tert-butyl hydroperoxide, cumene di(4-tert-butylcyclohexyl) hydroperoxide, cumene ethylperoxide, diisopropylhydroxy dicarboxylate, and combinations thereof.

The initiator used in this application is 0.2 to 5 weight % of the microcapsule composition.

In one non-limiting embodiment, a quencher is used during the preparation of the microcapsule. For example, 0.02 to 0.5 weight % of the microcapsule composition of ammonium or potassium peroxo disulfate is as quencher in this invention in the form of aqueous solution. Potassium peroxodisulfate is also known as potassium persulfate (K2S2O8). It is commonly used as an oxidizing agent and polymerization initiator in organic synthesis.

Particle Size of the Microcapsule compositions presented in this invention is ranging between 8-35 μm of Dv (90) value, as measured using a Malvern Mastersizer 3000.

### Application Products:

The Microcapsule compositions as per the present invention is suitable but not limited to applications in home and personal care products, textile products, in printing and coating applications, in pharmaceutical formulations, in consumer goods, and in agroindustrial formulation products.

Non-limiting examples of home care products containing Microcapsules according to the present inventions are categories into the followings: 1. Air care products, 2. House cleaners, 3. Dish washing products, 4. Laundering/fabric care products. Examples of air care products are broadly i) aqueous air freshener liquids, gels, sprays, upholstery refreshers & sprays, and liquids for metered dosing articles, ii) tablets, pellets, cakes, pastes, etc. 2. House cleaners with Microcapsule compositions according to this invention are multisurface cleaners, carpet cleaners, hard surface cleaners, etc. 3. Dishwashing products are liquid dishwashing agents, dishwashing tablets & cakes, etc. as per this application. 4. Laundering/fabric care products are majorly liquid and solid laundering detergents, fabric conditioners, fabric refreshers, fabric stiffeners, stain removing articles, fabric refresher sprays, solid fabric softener & refresher articles and fabric refresher cones, etc according to one embodiment of the present application.

Examples of personal care products with lipophilic core as active in microcapsules as per this present invention are leave on and rinse off hair and skin care compositions such as shampoo, conditioner, hair removal depilatory, hair styling gel, hair colorant, antiperspirant/deodorants, aqua mist, and spray-able & roll-on products, body washes, shower gels, hand washes, soap, body lotion, face washes, face mask, face cream, face serum, sunscreens, etc. Example of cosmetic products comprising off microcapsules such as but not limited to are lip-glossing articles, foundation, foundation primers, eyeshadow, etc.

Non-limiting examples of pharmaceutical formulations are primarily dermatological products such as ointments, sprays, creams, lotions, gels, and transdermal patches, etc.

Microcapsules with fragrances, anti-microbial and anti-viral agents as per this report are used in textile/fabric manufacturing to combat malodors and microbes. Additionally the microcapsules are applicated in the in diapers and sanitary napkins.

Printing formulations with microcapsules as per this application are used in inkjet printing, spraying, flexographic printing, roller and cylinder printings, stencil printing, digital printing, etc. where the lipophilic core of the microcapsules contains dyes and printing inks, fragrances, profragrances, anti-microbial and anti-viral agents, anti-malodor agents, etc. According to one embodiment of the present application, the coating materials for encapsulation are selected from the non-limiting examples where the core contains oil-soluble materials which have film-forming properties on skin and hair, such as vinyl pyrrolidone/hexadecene & vinyl pyrrolidone/eicosene copolymers, tricontanyl polyvinyl pyrrolidone, etc.

Non-limiting examples of consumer goods comprising off microcapsules according to this invention are paints, polishes for hard surfaces, shoe insoles, etc.

### EXAMPLES

### Example 1. Preparation of Microcapsule Composition as Per the Present Invention

### Raw Materials Used for the Preparation of the Aqueous Slurry of the Microcapsules

### Composition of Oil Phase 1:

Fragrance: 30 g (Lipophilic Core)
1a. Composition of Fragrance:

| Item | CAS No. | Weight % |
|---|---|---|
| Dihydromyrcenol | 18479-58-8 | 25 |
| Isononyl acetate | 40379-24-6 | 20 |
| Tetraahydro linalool | 78-69-3 | 15 |
| Nerolidol | 7212-44-4 | 10 |
| Hexyl acetate | 142-92-7 | 10 |
| Methyl hexyl ketone | 111-13-7 | 5 |
| Aldehyde C12 MNA | 110-41-8 | 5 |
| Lemonile | 61792-11-8 | 3 |
| Clonal | 2437-25-4 | 3 |
| Allyl amyl glycolate | 67634-00-8 | 2 |
| Manzanate | 39255-32-8 | 1.5 |
| Melonal | 106-72-9 | 0.5 |

Density of Fragrance is 0.8446 g/cm³ at 25° C. and the combined (averaged) Log P is 3.61
1b. Methacrylic acid: 2.2 g
1c. 2-Hydroxyethyl methacrylate: 0.65 g
1d. Ethylenediol dimethacrylate: 1.05 g
1e. Pentaerythritol tetraacrylate: 1.25 g
1f. Vinyl acetate: 0.61 g
1 g. Dilauroyl peroxide: 1.2 g

### Composition of Water Phase 2:

Water: 57.8 g
25% aqueous solution of sodium dodecyl sulfate: 8 g

### Composition of Water Phase 3:0.

13 g of Potassium peroxo disulphate in 10 g of water

### Process for Preparation of the Microcapsules

TABLE 1
Detailed Process for Preparation Microcapsule formulation as per this Invention

| Step no. | Process Details |
|---|---|
| 1 | Preparation of Oil Phase P1: Formation of prepolymer or oligomer |
| | 1.1. Addition of required amounts of different polymerisable molecules, thermal |
| | initiator in the lipophilic core |
| | 1.2. At about 40-55° C. under nitrogen stirring to be continued at 100-200 rpm |
| | for 15-45 minutes to form prepolymer(s). |
| 2 | Preparation of Water Phase P2 |
| | 2.1. Addition of desired quantity of aqueous solution of emulsifier (small |
| | molecule, polymer) in water |
| 3 | Preparation of Water Phase P3 |
| | 3.1. Addition of peroxo disulfate salt in water and dissolution of the salt by |
| | stirring at 250 rpm |
| 4 | Preparation of Emulsion |
| | 3.1. Addition of warm Phase P1 into Phase P2 |
| | 3.2. Emulsifying the oil phase of step 1 into the aqueous phase of step 2 by |
| | stirring at 500-1500 rpm for up to 12 minutes using a propeller type stirrer |
| 5 | Microencapsulation (Complete polymerization) |
| | 5.1. Heating the emulsion at 40-60° C. for 0.25 to 0.75 h under nitrogen while |
| | stirring at 100-400 rpm |
| | 5.2. Elevation of the temperature to 65-85° C. and to continue heating for another |
| | 4-6h under nitrogen while stirring at 100-400 rpm |
| | 5.3. Quenching the unreacted monomers at 65-85° C. by adding an aqueous |
| | peroxo disulfate solution (Phase 3) after 4-5 h of step 5.1 of Microencapsulation |

Comparative Examples 2-3. Preparation of Microcapsule compositions as per prior art with the present invention fragrance in order to allow olfactive properties comparison

TABLE 2
Comparative Examples 2-3 for Microcapsule compositions as per prior art

| Example | | Microcapsule Composition with |
|---|---|---|
| no. | Reference | Process of Microencapsulation |
| 2 | WO2017/123965 | An oil phase is prepared by adding 28.73 g of |
| | A1 (based on | fragrance of Example 1, followed by adding a |
| | ex. 1) | monomer mixture comprising off 4.16g of |
| | | pentaerythritol triacrylate (PETA) and 1.774 g |
| | | of methacrylic acid (MAA) to form an oil |
| | | mixture, wherein 0.17 g of 2,2′-Azobis(2- |
| | | methylpropionitrile) thermal initiator is |
| | | dissolved in the oil mixture under mechanical |
| | | stirring. Separately, an aqueous phase is |
| | | prepared by dissolving 2 g of Polyvinyl alcohol |
| | | into 65.15 g deionized water by heating to 80° C. |
| | | to form a polymer solution. The aqueous phase |
| | | is transferred into a 600 ml beaker and to this is |
| | | added the above prepared oil phase under a high |
| | | shear mixer to form an oil-in-water emulsion |
| | | (1200 rpm) with a mean droplet particle sizes of |
| | | around 20 microns. The formed oil-in-water |
| | | emulsion is transferred to a 50 0ml reaction |
| | | flask mounted on heat on blocks. The emulsion |
| | | is deoxygenated by bubbling nitrogen for 30 |
| | | minutes. After, the de-oxygenation with |
| | | nitrogen, the emulsion is heated up to 50 ° C. to |
| | | induce thermal polymerization for 30 min. |
| | | Then the temperature is increased to 80 ° C. for |
| | | total of 6 hours to form the microcapsule shell. |
| | | After 6 hours 0.06g of 2,2′-Azobis(2- |
| | | methylpropionitrile) is added consume any |
| | | residual monomers. |
| 3 | EP2397120B2 | Water phase (phase A) is prepared by adding |
| | (based on ex. 6) | 50.54 g of water and 10 g of 20% sodium |
| | | dodecyl sulfate solution. Oil phase (phase B) is |
| | | prepared by adding Fragrance of Example 1 |
| | | 24.52 g, Methacrylic acid 3.75 g, Methyl |
| | | methacrylate 1.32 g, 1,6-hexanediol |
| | | diacrylate 3.25 g and Dilauroyl peroxide 0.17 g |
| | | in a 250 ml closed stirred vessel fitted with an |
| | | anchor type stirrer. Phase B and then phase A |
| | | are added to the reactor and the agitator started |
| | | at time TO. The charge is processed to a finely |
| | | dispersed emulsion by adjusting the stirring |
| | | speed to 1100 rpm for 30 minutes at 35° C. while |
| | | a stream of nitrogen gas was bubbled through |
| | | the reaction mixture to remove oxygen. The |
| | | temperature was raised to 70° C. over the course |
| | | of 15 minutes and allowed to react for a further |
| | | 4 hours. After 3 hours 5 g of phase C |
| | | (Potassium peroxo diusulphate) added into the |
| | | reaction mixture. After 4 hours the reaction was |
| | | stopped whereby a microcapsule dispersion |
| | | was obtained. |

### Characterization of the Microcapsule Compositions:

### Particle Size Analysis:

Particle Size is analyzed using a Mastersizer 3000 with Hydro MV wet dispersion unit. Diluted aqueous solutions (0.5 weight %) of Microcapsule compositions of Example 1 and comparative examples 2-3 are measured and data analyzed using Mie scattering model and presented in Table 3.

TABLE 3
Dv (90) values of Microcapsule compositions
of Example 1 and comparative examples 2-3

| Microcapsule | |
|---|---|
| Composition | Dv (90) (in um) |
| Example 1 | 25.2 |
| Comp. Example 2 | 28.8 |
| Comp. Example 3 | 9.05 |

Percentage Free Oil Analysis after Microencapsulation:

1 g of Microcapsule composition slurry of Example 1 is mixed with 5 g of Hexane in a sealed tube. The mixture is shake it for 15 min in an orbital shaker at 300 rpm. Then the sample is allowed to rest for 10 min. The supernatant is passed through a 0.45 μm filter and subjected to GC analysis. The remaining fragrance is quantified using an Agilent INTOVU 9000 G3950A GC with a column Part No: 19091S-433UI-INT HP-5 MS UI 30m, 0.25 μm. The Microcapsule composition of Example 1 has almost no remaining free oil (0.13% of total core).

TABLE 4
Free oil analysis of Microcapsule compositions of
Example 1 and comparative examples 2-3

| Microcapsule | Percentage of free oil of |
|---|---|
| Composition | Microcapsule compositions |
| Example 1 | 0.13 |
| Comp. Example 2 | 0.45 |
| Comp. Example 3 | 2.25 |

This represents a very good indication of the superior encapsulation property of our microcapsule because it becomes more and more critical to develop consumer products which will release the active core material on demand without facing the potential drawbacks which could be generated by having (before use) excessive lipophilic material outside the microcapsule and inside the consumer product. This is beneficial for the end use of the microcapsules in consumer product as this ensure better post-rub performance of the microcapsules (due to efficient encapsulation of core fragrance) via the application through the consumer product, e.g., when the fabric treated with a fabric softener containing the microcapsules. The exceptional thermal stability of the microcapsules according to the invention enables the use of the microcapsules at higher temperature e.g., while steaming a fabric treated with product containing microcapsules.

### Olfactory Performance of Fragrance Core Microcapsules of Examples 1 and Comparative Examples 2-3

A regular fabric softening formulation is prepared in water with 5% Stepantex SP-90 as softening active and 0.1% sodium benzoate as preservative as suggested by the supplier (Stepan Company, Northbrook, IL 60062, United States). Separate fabric softening formulations are prepared using individual Microcapsule compositions of Example 1 and comparative examples 2-3 while maintaining the encapsulated fragrance core 0.3 weight % in the final softener formulations. Cotton fabrics are washed and treated with the softener formulations (dosage of 5 g of fabric softener formulation in 11 of water) and rinsed for 10 minutes before the fabrics are kept for drying at ambient conditions. The Olfactory profiles of the fabrics after drying are recorded by trained panel members for pre-rubbing and post-rubbing stages of the fabrics. Averaged results for the strength of the fragrances are presented in FIG. 1. The rating of strength varies from 1-5 where 1 is the weakest and 5 is being the strongest in the incremental scale. Example 1 exhibits the highest post-rubbing strength compared to the prior art microcapsule compositions at same level of loading of core (fragrance).

### Thermal Stability of the Microcapsule Compositions

### A. Heat Stability of the Microcapsule

Thermal Stability of the Microcapsules: Microcapsule composition is analyzed using Perkin TGA 4000 instrument where the slurry sample is heated from 30° C. to 500° C. at a ramp of 30° C./minute and then up to 800° C. at a ramp of 50° C./minute under nitrogen atmosphere. The major weight-losses in the thermogram are correlated with the associated phase changes in the microcapsule composition. A sharp drop in weight is noticed near 150-170° C. in the thermogram of FIG. 2 and this is associated majorly with the loss of water from the composition. The next weight-loss is associated with the breakage of shell and evaporation/loss of core (fragrance). The Microcapsule composition of Example 1 shows exceptional stability at around 250° C. and shows very minimum loss of weight. Therefore it clearly demonstrates the high thermal stability of the Microcapsule composition of present invention.

### B. Loss of Core Under High Temperature for Prolonged Time

Example 1 & comparative example 3 are investigated for loss of core under high temperature for prolonged time (30 min). This is to ensure the leakage of core at high temperature. Preventing loss of core at high temperature is a mandatory for applications of microcapsules at high temperature. Example 2 is not considered due to its low performance (as evidenced in FIG. 1). The sample is heated from 30° C. to 120° C. at a ramp of 3° C./minute and then it was kept at 120° C. for 30 minutes. The sample is further heated to 800° C. at a ramp of 50° C./minute under nitrogen atmosphere. The loss of weight is monitored with time at 120° C. for the microcapsule compositions and is presented in FIG. 3. It is evident from FIG. 3 that there is no loss weight in the Microcapsule composition of Example 1 within 30-60 minutes while the Microcapsules are exposed at 120° C. Whereas in Example 3 there is a significant loss of core at high temperature (nearly 10% loss of weight). Therefore no loss of core (fragrance) due to the shell-composition of the present invention. This also signifies the importance of present invention suitable for applications at higher temperatures.

### C. High Temperature Application

Steaming of Fabric applicated with fabric softener containing Microcapsules: Cotton fabric washed and treated with hereinabove detailed fabric softener containing Microcapsule composition of Example 1 of the present invention is subjected to hot steam for 3 minutes. Then after the fabric cool down to room temperature, pre-rubbing and post-rubbing evaluation of the fabric is conducted. The rating of strength varies from 1-5 as mentioned earlier where 1 is the weakest and 5 is being the strongest in the incremental scale. It is observed that after the steaming process, the strength of Microcapsules has been influenced almost negligibly as per the average post-rubbing value.

1. A microcapsule comprising a lipophilic core material and a microcapsule shell wherein said microcapsule shell is formed from oil-in-water emulsion polymerisation of a mixture of monomers, more than 50 percent by weight of the mixture of monomers consisting in monomers having a density higher than 1.05, said mixture of monomers comprising a) more than 30 percent by weight of one or more of ethylenically unsaturated acid monomer(s) based on the total weight of the mixture of monomers, b) one or more monofunctional acrylate and/or methacrylate monomer(s), c) one or more multifunctional acrylate and/or methacrylate monomers, and d) vinyl acetate monomer.

2. A microcapsule according to claim 1 which is thermally stable at 250° C.

3. A microcapsule according to claim 1 which is formaldehyde-free.

4. A microcapsule according to claim 1 wherein the log P values of all the monomers comprised in the mixture of monomers range from 0.5 to 4.0.

5. A microcapsule according to claim 1 wherein the ethylenically unsaturated acid monomer(s) is selected from acrylic acid, methacrylic acid, crotonic acid, 2-carboxyethyl acrylate, glutaconic acid, 3,3-Dimethylacrylic acid, itaconic acid, maleic acid, fumaric acid or a mixture of two or more of said acids.

6. A microcapsule according to claim 1 wherein the ethylenically unsaturated acid monomer is methacrylic acid.

7. A microcapsule according to claim 5 wherein the concentration of ethylenically unsaturated acid monomers in the monomer mixture is equal or inferior to 45 percent by weight based on the total weight of the mixture of monomers.

8. A microcapsule according to claim 5 wherein the concentration of ethylenically unsaturated acid monomers in the monomer mixture is comprised between 30 and 45 percent by weight based on the total weight of the mixture of monomers.

9. A microcapsule according to claim 1 wherein the monofunctional acrylate and/or methacrylate monomer(s) is selected from polymerizable molecules with one ester functionality of the following formula 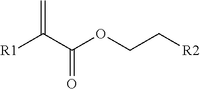
wherein
R1═H/CH₃,
R2═—OH, —(CH₂)ₙ—OH, O—CH₃, —O—(CH₂)ₘ—OH, —O—(CH₂)ₙ—CH₃, —(O—CH₂—CH₂)ₙ—OH, —(O—CH₂—CH₂—CH₂)ₙ—OH, —(O—CH₂—CH₂)ₙ—O—CH₃, —(O—CH₂—CH₂)ₙ—O—CH₂—CH₃, —(O—CH₂—CH₂—CH₂)ₙ—O—CH₂—CH₃, —(O—CH₂—CH₂—CH₂)ₙ—O—CH₃, —(O—CH₂—CHR3)ₙ—CH₃
n=1 to 10, m=2 to 10, and R3=methyl or ethyl,
or a mixture of two or more of said monomers.

10. A microcapsule according to claim 1 wherein the monofunctional acrylate and/or methacrylate monomer(s) is selected from 2-hydroxyethyl methacrylate, poly(ethylene glycol) methacrylate, Poly(propylene glycol) methacrylate, 4-hydroxybutyl acrylate, hydroxybutyl methacrylate, Hydroxypropyl acrylate, Hydroxypropyl methacrylate 6-hydroxyhexyl acrylate, 6-hydroxyhexyl methacrylate, or a mixture of two or more of said monomers.

11. A microcapsule according to claim 1 wherein the monofunctional acrylate and/or methacrylate monomer(s) is hydroxyethyl methacrylate.

12. A microcapsule according to claim 9 wherein the concentration of the monofunctional acrylate and/or methacrylate monomer(s) is comprised between 5 and 50 percent by weight based on the total weight of the mixture of monomers.

13. A microcapsule according to claim 1 wherein the multifunctional acrylate and/or methacrylate monomer(s) is selected from polymerizable molecules with more than one ester functionalities.

14. A microcapsule according to claim 1 wherein the multifunctional acrylate and/or methacrylate monomer(s) is selected from ethylene glycol dimethacrylate, neopentyl glycol dimethacrylate, 1,4-Butanediol diacrylate, 1,4-Butanediol dimethacrylate, 1,6-hexane diol dimethacrylate, Glycerol diacrylate, Glycerol dimethacrylate, 1,10-Decanediol dimethacrylate, Bis [2-(methacryloyloxy)ethyl]phosphate, pentaerythritol triacrylate, trimethylolpropane triacrylate, trimethylolpropane trimethacrylate, pentaerythritol tetraacrylate, or a mixture of two or more of said monomers.

15. A microcapsule according to claim 14 wherein the multifunctional acrylate and/or methacrylate monomer(s) is a mixture of two or more of said monomers, each of said monomer representing less than 30 percent by weight based on the total weight of the mixture of monomers.

16. A microcapsule according to claim 13 wherein the total concentration of the multifunctional acrylate and/or methacrylate monomer(s) in the monomer mixture is less than 60 percent by weight based on the total weight of the mixture of monomers.

17. A microcapsule according to claim 13 wherein the concentration of the multifunctional acrylate and/or methacrylate monomer(s) in the monomer mixture is comprised between 20 and 50 percent by weight based on the total weight of the mixture of monomers.

18. A microcapsule according to claim 1 wherein the concentration of vinyl acetate monomer in the monomer mixture is comprised between 0.05 and 15 percent by weight based on the total weight of the mixture of monomers.

19. A microcapsule according to claim 1 wherein the concentration of a) ethylenically unsaturated acid monomer(s), b) monofunctional acrylate and/or methacrylate monomer(s), c) multifunctional acrylate and/or methacrylate monomer(s), and d) vinyl acetate monomer, is at least 95 percent by weight based on the total weight of the mixture of monomers.

20. A microcapsule according to claim 19 wherein the concentration of [a)+b)+c)+d)] is 100 percent by weight based on the total weight of the mixture of monomers.

21. A microcapsule according to claim 1 wherein the Particle Size of the microcapsule ranges from 8 to 35 microns of Dv (90) value.

22. A microcapsule according to claim 1 wherein the lipophilic core material of the microcapsules has a density equal or less than 0.95 g/cm³ at 25° C. with a combined log P comprised between 2.5 and 6.0.

23. A microcapsule according to claim 1 wherein the lipophilic core material of the microcapsules comprises at least 95 percent by weight based on the total weight of said lipophilic core material, of one or more of the following ingredients: fragrances, profragrances, emollient oils, essential oils, hair-benefitting agents, skin-benefitting agents, conditioner actives, cosmetic care actives, personal care actives, UV absorbers, vitamins, anti-oxidants, anti-microbial agents, anti-viral, flavors, anti-malodor agents, pharmaceutical agents, dyes, printing inks, pesticides, biocides, agrochemicals, coating materials, anti-ageing actives.

24. A microcapsule according to claim 23 wherein the lipophilic core material of the microcapsules comprises one or more of the following ingredients: fragrances, essential oils, hair-benefitting agents, skin-benefitting agents, anti-microbial agents, anti-viral agents, anti-malodor agents.

25. A microcapsule according to claim 1 wherein the lipophilic core material weight divided by the shell weight of the microcapsule is comprised between 15 and 0.33.

26. Aqueous microcapsule composition comprising water and microcapsules according to claim 1 wherein the water represents from 35 to 82 weight percent of the total weight of the aqueous microcapsule composition.

27. Aqueous microcapsule composition according to claim 26 wherein the lipophilic core material represents between 15 and 45 percent by weight of the total weight of the aqueous microcapsule composition.

28. Aqueous microcapsule composition according to claim 26 comprising one or more emulsifiers wherein the emulsifiers represent from 0.05 to 5 weight percent of the total weight of the aqueous microcapsule composition.

29. Aqueous microcapsule composition according to claim 28 wherein the weight of water, microcapsules and emulsifier represents at least 90% by weight of the total weight of the aqueous microcapsule composition.

30. A process for preparing an aqueous microcapsule composition as claimed in claim 26 comprising the steps of:
1. dissolving the mixture of monomers together with an initiator in an oil phase comprising the lipophilic core material and heating the oil phase to form prepolymer(s),
2. dissolving an emulsifier in an aqueous phase,
3. emulsifying the oil phase of step 1 into the aqueous phase of step 2, and
4. heating the emulsion from step 3 to form a suspension of core-shell microcapsules in water.

31. A process according to claim 30 wherein the emulsification step of the core phase in the water phase is obtained by stirring at 500-1500 rpm for up to 12 minutes using a propeller type stirrer.

32. A non-therapeutic method of use of a microcapsule according to claim 1 or an aqueous microcapsule composition thereof comprising employing said microcapsule to deliver the lipophilic core material for industrial compositions that are related to home care products, personal care products, textile products, printing and coating applications products, pharmaceutical formulations products, consumer goods products, and in agro-industrial formulation products.

33. Non-therapeutic method of use according to claim 32 wherein the mechanical stress and temperature conditions at which the microcapsule is exposed are sufficient to break the microcapsule shell and to deliver the lipophilic core material.

34. Non-therapeutic method of use according to claim 32 for the steaming of fabric, hair-straightening, paints, textile-processing, and shoe insole making.
